(19) United States
(12) Patent Application Publication
(10) Pub. No.: US 2021/0085406 A1
(43) Pub. Date: Mar. 25, 2021

(54) USER INTERFACE FOR A SURGICAL ROBOTIC SYSTEM

(71) Applicant: TransEnterix Surgical, Inc., Morrisville, NC (US)

(72) Inventors: Dustin Owen Vaughan, Raleigh, NC (US); Anthony Fernando, Chapel Hill, NC (US); Alexander John Maret, Apex, NC (US); Paul Wilhelm Schnur, Pipersville, PA (US); Stephan Atay, Raleigh, NC (US)

(21) Appl. No.: 17/114,467

(22) Filed: Dec. 7, 2020

Related U.S. Application Data
(63) Continuation-in-part of application No. 16/732,945, filed on Jan. 2, 2020.
(60) Provisional application No. 62/944,596, filed on Dec. 6, 2019.
(60) Provisional application No. 62/787,304, filed on Jan. 1, 2019.

(51) Int. Cl. A61B 34/30; A61B 34/00

(52) U.S. Cl. CPC A61B 34/30; A61B 90/361; A61B 2034/742; A61B 34/74

(57) ABSTRACT

A surgical robotic system has at least two robotic manipulator arms. A user input device has a single handle configured to allow a surgeon to input instructions to the robotic surgical system to cause movement of the first instrument by a first robotic manipulator arm, and to cause movement of the second instrument by a second robotic manipulator arm. The user input device can include a first input handle moveable in multiple degrees of freedom to generate input for movement of the first instrument, and an auxiliary input on the handle to generate input for movement of the second instrument.

This application claims the benefit of U.S. Provisional Application No. 62/944,596, filed Dec. 6, 2019. This application is also a continuation in part of U.S. application Ser. No. 16/732,945, filed Jan. 2, 2020, which claims the benefit of U.S. Provisional Application No. 62/787,304, filed Jan. 1, 2020.

### TECHNICAL FIELD OF THE INVENTION

The present invention relates generally to the field of surgical robotic systems.

### BACKGROUND

Surgical robotic systems use one or more robotic manipulators or robotic arms. Each manipulator carries a surgical instrument, or the camera used to capture images from within the body for display on a monitor. Typical configurations allow two or three instruments and the camera to be supported and manipulated by the system. Input to the system is generated based on input from a surgeon positioned at a surgeon console, typically using input devices such as input handles and a foot pedal. Motion and actuation of the surgical instruments and the camera is controlled based on the user input. The image captured by the camera is shown on a display at the surgeon console. The console may be located patient-side, within the sterile field, or outside of the sterile field.

The console may include two input devices which can be gripped by the surgeon and moved so as to deliver instructions to the system as to the desired movement and operation of the instruments supported by the robotic arms. The surgeon's movements are suitably reproduced by the surgical instruments by means of movement of the robotic arms. The input devices may be equipped to provide the surgeon with tactile feedback so that the surgeon can feel on the input devices the forces exerted by the instruments on the patient's tissues.

Although the inventions described herein may be used on a variety of robotic surgical systems, the embodiments will be described with reference to a system of the type shown in FIG. 1. In the illustrated system, a surgeon console 12 has two input devices such as handles 17, 18. The input devices 12 are configured to be manipulated by a user to generate signals that are used to command motion of a robotically controlled device in multiple degrees of freedom. In use, the user selectively assigns the two handles 17, 18 to two of the robotic manipulators 13, 14, 15, allowing surgeon control of two of the surgical instruments 10a, 10b, and 10c disposed at the working site at any given time. To control a third one of the instruments disposed at the working site using a handle, one of the two handles 17, 18 is operatively disengaged from one of the initial two instruments and then operatively paired with the third instrument. A fourth robotic manipulator, not shown in FIG. 1, may be optionally provided to support and maneuver an additional instrument.

One of the instruments 10a, 10b, 10c is a camera that captures images of the operative field in the body cavity. The camera may be moved by its corresponding robotic manipulator using input from a variety of types of input devices, including, without limitation, one of the handles 17, 18, additional controls on the console, a foot pedal, an eye tracker 21, voice controller, etc. The console may also include a display or monitor 23 configured to display the images captured by the camera, and for optionally displaying system information, patient information, etc.

A control unit 30 is operationally connected to the robotic arms and to the user interface. The control unit receives user input from the input devices corresponding to the desired movement of the surgical instruments, and the robotic arms are caused to manipulate the surgical instruments accordingly.

The input devices 17, 18 are configured to be manipulated by a user to generate signals that are processed by the system to generate instructions used to command motion of the manipulators in order to move the instruments in multiple degrees of freedom. As described in application US 2013/0012930, the ability to understand the forces that are being applied to the patient by the robotically controlled surgical devices during minimally invasive surgery is highly advantageous to the surgeon. Communication of information representing such forces to the surgeon via the surgeon interface is referred to as “haptic feedback.” In some systems, haptic feedback is communicated to the surgeon in the form of forces applied by motors to the surgeon interface, so that as the surgeon moves the handles of the surgeon interface, s/he feels resistance against movement representing the direction and magnitude of forces experienced by the robotically controlled surgical device. Forces represented can include both the forces at the tips of the robotically controlled devices and/or the forces being applied by the shaft of the robotically controlled device to the trocar at the entrance point to the body, giving the surgeon complete understanding of the forces applied to the device so s/he can better control the device during surgery.

The surgical system allows the operating room staff to remove and replace the surgical instruments 10a, b, c carried by the robotic manipulator, based on the surgical need. When an instrument exchange is necessary, surgical personnel remove an instrument from a manipulator arm and replace it with another.

The number of degrees of freedom (DOFs) of motion for a robotically controlled instrument can vary between surgical systems and also between the different devices used for a particular system. Likewise, instruments with varying levels of complexity can be used interchangeably on a particular type of robotic system.

For example, a robotically controlled rigid-shafted instrument that moves similarly to a conventional laparoscopic instrument will be pivoted by the robotic arm relative to a fulcrum at the incision site (instrument pitch-yaw motion), axial roll of the instrument about its longitudinal axis, and translation along the longitudinal axis of the instrument (along the axis of insertion/withdrawal of the instrument relative to the incision). A user input device designed to give instruments for movement and actuation of this type of instrument can be fairly simple. For example, FIG. 2 shows a grip 50 that might be used on a user input device to move that type of simple robotically manipulated laparoscopic instrument. The grip 50 is very similar to a simple laparoscopic instrument grip, and it works with the remaining features of the user input device (linkages and/or gimbals, for example) so that the user directions movement of the surgical instrument using hand movements familiar to the laparoscopic surgeon. For example, the grip is pivoted in one direction to pivot the instrument tip in the opposite direction within the body, so that pivoting the instrument handle downwardly causes the robotic system to pivot the instrument tip upwardly, etc. The user advances/retracts the grip along its longitudinal axis to cause the robotic arm to advance/retract the instrument along its insertion axis. Pivoting one or two grip members 52 relative to the grip 50 is used as input to open/close the jaws of the instrument.

A robotically controlled rigid-shafted instrument that moves similarly to a conventional laparoscopic instrument having slightly more complexity than that described in the prior paragraph might require a slightly more complex grip for the user input devices. If, for example, the instrument adds a degree of articulation of its end effector about its shaft, and/or the ability to axially roll the instrument's tip about the shaft, a grip can be used to facilitate use of those features. In the example shown in FIG. 3, instrument pitch-yaw motion, instrument roll and insertion axis motion and jaw open-close can be achieved by moving the grip 54 in the same ways discussed with respect to the second embodiment. In addition, axial tip roll can be achieved by rotating a knob 56 or lever on the grip 54 (e.g. using the index finger), and movement of the degree of articulation can be commanded by pivoting the grip about pivot axis P.

Handles incorporating additional degrees of freedom might be needed for surgical instruments having greater complexity. For example, an instrument that includes an elongate rigid shaft having a region that can be robotically controlled to articulate or bend can have additional DOFs in the region of the articulation or bend. As a more specific example, such an instrument might be configured to move the instrument tip or end effector in pitch and/or yaw relative to the instrument shaft (i.e. in addition to the pitch and/or yaw that results from movement of the rigid instrument shaft about a fulcrum at the incision site), giving the instrument 6DOFs. See, for example, the instruments described in co-pending and commonly owned application U.S. Ser. No. 16/732,306, Articulating Surgical Instrument.

There are other types of user instrument handle motion, besides laparoscopic motion, used in surgery. Another type of instrument handle motion used in surgery is referred to as “true cartesian motion,” which differs from laparoscopic motion in that there is no inversion of the motion, so the user input handle is raised to cause the surgical robotic system to raise the instrument tip, moved left to cause movement of the tip to the left, etc. Some surgical systems may allow surgical personnel to choose whether the system will operate in a laparoscopic type of mode or in a true cartesian motion mode. Others might make use of a surgeon console that is configured so it can be selectively used use with a laparoscopic surgical system and with a true cartesian surgical system.

One system of the type described above and shown in FIG. 1 is the Senhance® Surgical System marketed by TransEnterix, Inc., Morrisville, N.C.

When two of the arms are under control of the user input devices 17, 18, it may be desirable for the user to perform a task using a third one of the arms without having to re-assign a user input device 17, 18 to that arm. This application describes a configuration in which one of the user input devices 17, 18 may be configured to cause movements of two of the robotic arms without requiring re-assignment.

### BRIEF DESCRIPTION OF THE DRAWINGS

FIG. 1 is a schematic view of a robotic surgery system.

FIG. 2 shows a user input device of the system of FIG. 1.

FIG. 3 is a perspective view of an alternative user input device, including a portion of a linkage interface between the handle and the surgeon console;

FIG. 4 is a side elevation view of the handle shown in FIG. 3;

FIG. 5 is a read elevation view of the handle shown in FIG. 4.

### DETAILED DESCRIPTION

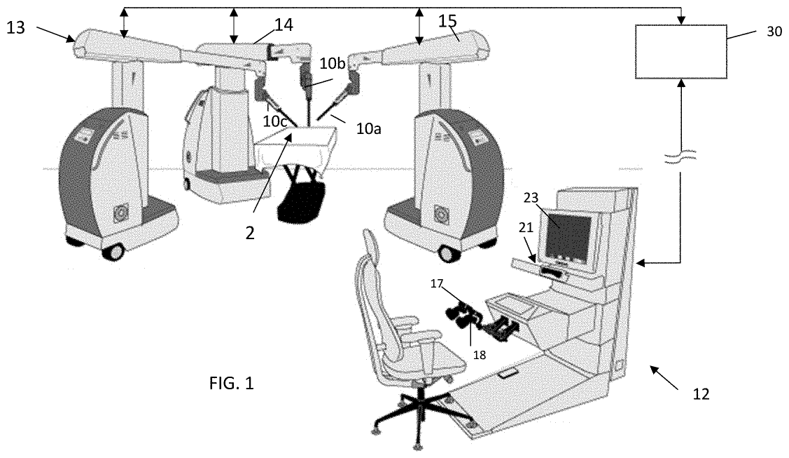

In a surgical robotic system, which may be of the type shown in FIG. 1 or an alternate system, a user input device may configured to provide motion of the robotic arm with which that input device has been paired, and (as applicable) actuation of the surgical instrument carried by that arm. The user interface device 60 is preferably part of a surgeon console for a robotic surgical system (see, for example, surgeon console 12 of FIG. 1). The concepts described here are suitable for use with any type of user interface device that is manipulated by a user to generated input to a surgical robotic system for manipulation of a corresponding surgical instrument. Examples include the user interface of the surgeon console of the Senhance Surgical System marketed by TransEnterix, Inc., Morrisville, N.C., a user interface device of the type described in co-pending and commonly owned U.S. application Ser. No. 16/513,670 (“Haptic User Interface for Robotically Controlled Surgical Instruments”) and any other type of interface.

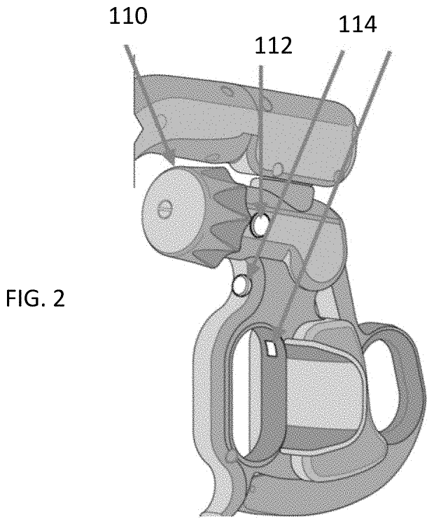

For example, the user input is used to cause movement of the arm, or a portion of the arm, that moves the instrument along the insertion axis, in pitch and yaw, and/or to axially roll the instrument about its axis. In a user input device configuration such as is shown in FIGS. 1 and 2, user input motions follow those a surgeon would use when manipulating a laparoscopic surgical instrument, i.e. insertion movement is achieved by moving the input device handle to simulate insertion or withdrawal of the instrument, pitch and yaw motion are achieved by moving the handle up-down or side-side, and roll motion may be achieved by rotating a finger knob (see the knob 110 shown in FIG. 2). Opening and closing of the jaws, if applicable, can be achieved by opening and closing the hand grips of the user input device.

The input device may additionally include features that allow a surgeon to cause movement of a third one of the arms while that input device remains set-up to control the first or second arm. For example, the input devices 17, 18 might be in control of the arms holding instruments, but one of the input devices might additionally be used to cause movement of the camera. As one example, an input (e.g. button 112, 114) on one of the input devices may be pressed to causing the arm holding the camera to move the camera in and/or out along the insertion axis. Two such inputs may be provided, one for inward movement and other for outward movement. Alternatively, this or an alternative input device might incorporate a joystick. A joystick on one of the input devices can allow more complex motions, such as movement of the arm holding the camera (or third instrument) in multiple degrees of freedom, or articulation of the camera or third instrument. In the latter example, the camera or third instrument might include joints or bend regions along the shaft or at the end effector, with the articulation occurring at the joints or bend regions. In this manner, a single handle of a user input device may be used to direct independent movement of two instruments carried by independently moveable robotic arms.

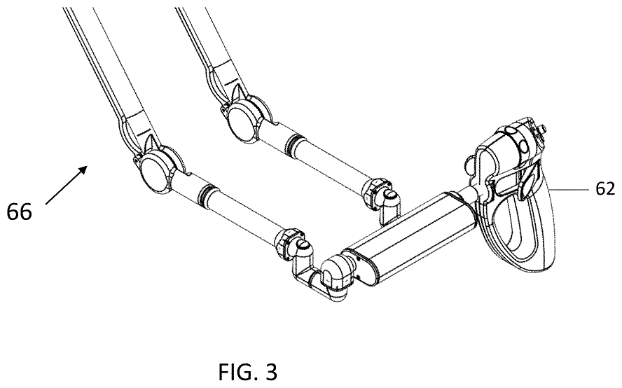

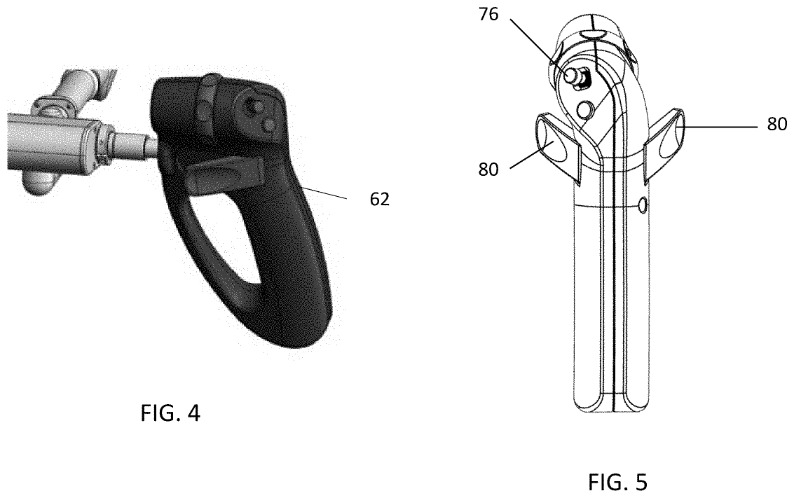

Although the FIG. 2 embodiment might include a mechanical or optical joystick, FIG. 3 illustrates another example of a user interface device which includes a joystick 76. In this embodiment, the interface assembly 66 or linkage coupling the handle to the console (a portion of which is shown) is the type described in co-pending U.S. application Ser. No. 16/513,670 (“Haptic User Interface for Robotically Controlled Surgical Instruments”), but alternative interface assemblies may be used. The handle 62 can having one of a variety of degrees of complexity, sizes and/or shapes, motion types (e.g. laparoscopic motion or true cartesian motion), grip configurations (e.g. scissor grip, pistol grip, etc.) or jaw actuation mechanisms (e.g. scissor handle type arrangements, two- or one-lever mechanisms, triggers, finger loops, paddle arrangements (described in connection with FIG. 5.

Some handles may incorporate tactile (e.g. vibratory) motors and/or brushed/brushless DC motors for haptic feedback.

In one configuration in which a surgical instrument having pitch and jaw articulation at its distal end (e.g. one of the type described in U.S. Ser. No. 16/732,306, entitled Articulating Surgical Instrument), movement of the handle itself will control instrument yaw and pitch motion etc., while the joystick will be moved by the user (e.g. thumb control) to cause pitch/yaw articulating at the end effector). Some embodiments might include a center click/selection of the joystick similar to that found on consumer gaming systems. This additional input capacity could be used for locking the wrist in its configured orientation or resetting the wrist back to a home/straight position. The joystick would allow very simple and standard 4 DOF control of a laparoscopic instrument but also enable the user to quickly and instinctively set the end effector position distal to a 2 DOF wrist segment. The user could then continue to operate the instrument in typical laparoscopic fashion while retaining that set wrist position. This is considered static control of the wrist segment as the user is not continuously having to maintain that wrist orientation relative to the shaft. Those elements of the instrument are only actuated/moved under direct instruction from the user.

In other embodiments, as described previously, the joystick is used to control movement of another surgical instrument, such as the camera that is positioned on the body. This may control laparoscopic movement of the camera (as moved by the robotic arm supporting it), or articulation/bending at the distal end of the instrument. The system may be configured so the user can select between these or other available functions for the joystick. Other uses for the joystick include the following:
Menu selection functions.
Navigation to and selection of icons, menu items, etc. displayed to the user on a monitor, such as the image display (i.e. control a mouse cursor)
Controlling insertion axis motion (moving an instrument axially into/out of patient using the robotic manipulator)
Clutching instruments (suspension of the control relationship between handle motion and instrument)
Changing operational settings for the system (e.g. motion scaling, enabling/disabling features such as turning on/off eye-tracking camera control which may be of the type described in commonly owned U.S. patent Ser. No. 10/251,713)

All patents and applications referred to herein, including for purposes of priority, are incorporated herein by reference.

What is claimed is:

1. A surgical robotic system comprising:
at least two robotic manipulator arms;
a first instrument on a first one of the robotic manipulator arms;
a second instrument on a second one of the robotic manipulator arms; and
a user input device comprising a single handle configured to allow a surgeon to input instructions to the robotic surgical system to cause movement of the first instrument by the first robotic manipulator arm, and to cause movement of the second instrument by the second robotic manipulator arm.

2. The system of claim 1, wherein the user input device includes a first input handle moveable in multiple degrees of freedom to generate input for movement of the first instrument, and an auxiliary input on the handle to generate input for movement of the second instrument.
